(19) United States
(12) Patent Application Publication
(10) Pub. No.: US 2019/0298743 A1
(43) Pub. Date: Oct. 3, 2019

(54) TREATMENT OF PAIN

(71) Applicants: Takeda Pharmaceutical Company Limited, Osaka (JP); MONASH UNIVERSITY, Clayton, Victoria (AU); QUEENS UNIVERSITY, Kingston (CA)

(72) Inventor: Nigel Bunnett, Elwood (AU)

(73) Assignee: Takeda Pharmaceutical Company Limited, Osaka (JP)

(21) Appl. No.: 16/303,023

(22) PCT Filed: May 19, 2017
(86) PCT No.: PCT/AU2017/050469

§ 371 Date: Nov. 19, 2018

(30) Foreign Application Priority Data
May 20, 2016 (AU) 2016901912

(51) Int. Cl. A61K 31/58; A61P 23/00

(52) U.S. Cl. CPC A61K 31/58; A61P 23/00

(57) ABSTRACT

The present invention relates to compounds and their uses. In particular, to compounds that inhibit endosomal protease-activated receptor-2 (PAR₂) signaling and their use in the treatment of pain.

### FIELD OF THE INVENTION

The present invention relates to compounds and their uses. In particular, to compounds that inhibit endosomal protease-activated receptor-2 (PAR₂) signaling and their use in the treatment of pain.

### BACKGROUND OF THE INVENTION

G protein-coupled receptors (GPCRs) are the largest family of cell-surface receptors, participate in most pathophysiological processes, and are the target of ˜30% of therapeutic drugs (Audet, M. & Bouvier, M. Nat Chem Biol 2008, 4, 397-403). Cell-surface GPCRs interact with extracellular ligands and couple to heterotrimeric G proteins, which trigger plasma membrane delimited signals (second messenger formation, growth factor receptor transactivation, ion channel regulation). Ligand removal and receptor association with β-arrestins (βarrs) terminate plasma membrane signals.

Until recently, it was widely assumed that activation of GPCRs, subsequent down stream signaling and signal termination took place exclusively at the plasma membrane. Plasma membrane signaling is terminated within minutes of activation via phosphorylation of the receptor by GPCR kinases (GRKs) that are selective for the active ligand-bound receptor conformation. GRKs phosphorylate C-terminal S/T-rich domains of GPCRs (Sato, P. Y., et al., Physiological reviews 2015, 95, 377-404). Phosphorylated receptors then bind to βarr, which sterically prevents coupling between receptor and G-protein, thus terminating agonist-mediated G-protein activation. βarrs further promote the transfer of ligand-bound receptor from the cell surface to early endosomes via dynamin- and clathrin-dependent endocytosis. Once endocytosed, the ligand and phosphate groups are removed from the GPCR and the receptor is either rapidly redistributed to the cell membrane or it is transported to a lysosome for degradation.

Recently, however, it has been discovered that a diverse range of GPCRs do not always follow the conventional paradigm. Studies have found that following ligand binding and activation of the receptor, some cell surface GPCRs internalise and redistribute into early endosomes where heterotrimeric G protein signaling is maintained for an extended period of time. Accordingly, rather than merely acting as a conduit for GPCR trafficking to recycling or degradatory pathways, endosomes can be a vital site of signal transduction (Murphy, J. E. et al. Proc Natl Acad Sci USA 2009, 106, 17615-17622). By recruiting GPCRs and mitogen-activated protein kinases to endosomes, βarrs can mediate endosomal GPCR signaling (Murphy, J. E. et al. Proc Natl Acad Sci USA 2009, 106, 17615-17622; DeFea, K. A. et al. Proc Natl Acad Sci USA 2000, 97, 11086-11091; DeFea, K. A. et al. J Cell Biol 2000, 148, 1267-1281).

βarrs recruit diverse signaling proteins to activated receptors at plasma and endosomal membranes and are essential mediators of signaling. The MAPK cascades [ERK, c-Jun amino-terminal kinase (JNK), p38] are the most thoroughly characterized βarr-dependent signaling pathways. The first evidence that βarrs are active participants in signaling was the observation that dominant negative mutants of βarr inhibited β₂AR-induced activation of ERK1/2 (Daaka Y, et al. J Biol Chem 1998, 273, 685-688). Subsequently, βarrs were found to couple β₂AR to c-Src and mediate ERK1/2 activation (Lutterall L. M. et al. Science 1999, 283, 655-661). βarrs similarly participate in ERK1/2 signaling by other GPCRs, including neurokinin-1 receptor (NK₁R), protease-activated receptor 2 (PAR₂), angiotensin II type 1A receptor (AT_{1A}R), and vasopressin V2 receptor (V₂R). These observations led to the view that βarrs are scaffolds that couple activated GPCRs with MAPK signaling complexes. βarrs thereby mediate a second wave of GPCR signaling that is distinct from G protein-dependent signaling at the plasma membrane.

### SUMMARY OF THE INVENTION

The present invention is predicated on the discovery that inhibiting endosomal signaling of PAR₂ can provide a novel method for the treatment of protease-evoked pain. Protease-activated receptor-2 (PAR₂) is a major mediator of protease-evoked inflammation and pain. PAR₂ is expressed by primary sensory neurons that control neurogenic inflammation and pain transmission. Multiple proteases that are generated during injury and inflammation can activate PAR₂ on sensory nerves, including epithelial derived trypsin IV, mast cell tryptase, macrophage cathepsin S and neutrophil elastase. These proteases cleave and activate PAR₂ on primary sensory nerves, leading to sensitization of transient receptor potential ion channels and the release of neuropeptides (substance P and calcitonin gene related peptide). These peptides cause peripheral inflammation and central transmission of pain. Trypsin and tryptase activate PAR₂ by canonical mechanisms, leading to recruitment of β-arrestins and PAR₂ endocytosis.

It has now been discovered that inhibiting endosomal signaling of PAR₂, by either preventing endocytosis of the activated receptor or by targeting and inhibiting endosomal PAR₂ signaling, provides a novel method for the treatment of protease-evoked pain.

Accordingly, in one aspect the present invention provides a method for the treatment of protease-evoked pain comprising administering to a subject in need thereof a compound that inhibits endosomal signaling of protease-activated receptor-2 (PAR₂).

In one aspect, the compound that inhibits endosomal PAR₂ signaling is a compound that inhibits endocytosis of the activated receptor.

In another aspect, the compound that inhibits endosomal PAR₂ signaling is a compound that targets and inhibits endosomal PAR₂ signaling.

In a further aspect, the compound that inhibits endosomal PAR₂ signaling is a tripartite compound that inhibits endosomal PAR₂ signaling of formula (I):

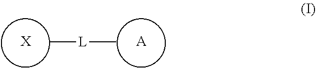

wherein
A is a lipid anchor that promotes insertion of the compound into a plasma membrane;
L is a linker moiety of 1 nm to 50 nm in length; and
X is an inhibitor of endosomal PAR₂ signaling;
wherein the lipid anchor partitions into lipid membranes that are insoluble in non-ionic detergent at 4° C.; or
a pharmaceutically acceptable salt thereof.

In another aspect, the present invention provides method for the treatment of protease-evoked pain comprising administering to a subject in need thereof a combination comprising one or more compounds that inhibit endocytosis of the receptor and one or more compounds that target and inhibit endosomal PAR₂ signaling.

In another aspect, the present invention provides use of a compound that inhibits endosomal signaling of PAR₂ in the manufacture of a medicament for the treatment of protease-evoked pain.

In a further aspect, the present invention provides a compound that inhibits endosomal signaling of PAR₂ for use in the treatment of protease-evoked pain.

These and other aspects of the present invention will become more apparent to the skilled addressee upon reading the following detailed description in connection with the accompanying examples and claims.

### BRIEF DESCRIPTION OF THE DRAWINGS

FIG. 1: BRET assays with plasma membrane marker, RIT-venus. (a) ΔBRET kinetics upon stimulation by three known agonists of PAR₂. (b) Trypsin CRC (produced from AUC data). (c) Change in trypsin induced trafficking upon 30 minute pre-treatment with various endocytic inhibitors and control compounds. Data are presented as means+SEM from at least three independent experiments, each containing three replicates per condition.

FIG. 2: BRET assays with early endosomal marker, Rab5a-venus. (a) ΔBRET kinetics upon stimulation by three known agonists of PAR₂. (b) Trypsin CRC (produced from AUC data). (c) Change in trypsin induced trafficking upon 30 minute pre-treatment with various endocytic inhibitors and control compounds. Data are presented as means+SEM from at least three independent experiments, each containing three replicates per condition.

FIG. 3: FRET assays with cytosolic ERK sensor, CytoEKAR. (a) ΔFRET kinetics upon stimulation with two known agonists of PAR₂. (b) Trypsin CRC (produced from AUC data). (c) Change in trypsin induced signaling upon 30 minute pre-treatment with various endocytic inhibitors and control compounds. Data are presented as means+SEM from at least three independent experiments, each containing three replicates per condition.

FIG. 4. FRET assays with nuclear ERK sensor, NucEKAR. (a) ΔFRET kinetics upon stimulation with two known agonists of PAR₂. (b) Trypsin CRC (produced from AUC data). (c) Change in trypsin induced signaling upon 30 minute pre-treatment with various endocytic inhibitors and control compounds. Data are presented as means+SEM from at least three independent experiments, each containing three replicates per condition.

FIG. 5. BRET resensitisation assays. (a) ΔBRET kinetics upon stimulation with three known agonists of PAR₂. (b) Change in protease induced trafficking patterns upon 30 minute pre-treatment with various signaling inhibitor. Data are presented as means+SEM from at least two independent experiments, each containing two replicates per condition.

FIG. 6. Neuronal excitation of isolated DRG neurons and inhibition thereof with inhibitors of endocytosis of PAR₂.

FIG. 7. Neuronal excitation of isolated DRG neurons and inhibition thereof with inhibitors of endocytosis of PAR₂.

FIG. 8. Inhibition of trypsin-evoked mechanical hyperalgesia in mice.

FIG. 9. Inhibition of trypsin-evoked mechanical hyperalgesia in mice.

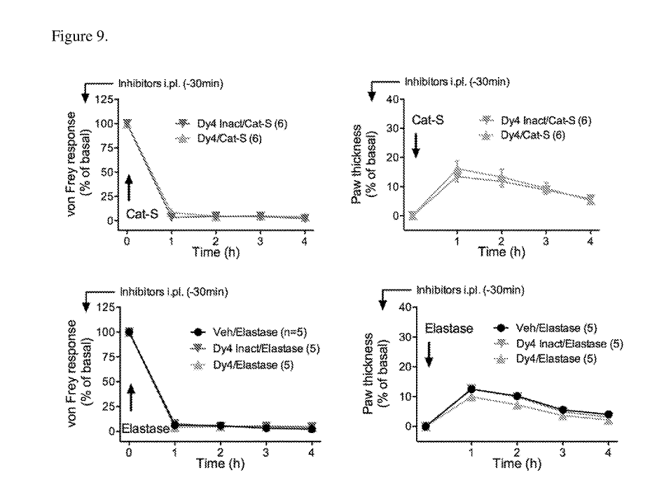

### DETAILED DESCRIPTION OF THE INVENTION

The contribution of endocytosis of protease-activated receptor-2 (PAR₂) was evaluated for generation of signals in subcellular compartments that mediate protease-evoked sensitization of nociceptors. Bioluminescence resonance energy transfer (BRET) was used to assess the proximity of PAR₂ to resident proteins of the plasma membrane (KRas) and endosomes (Rab5a) in HEK cells. The canonical agonist trypsin, which cleaves PAR₂ at R³⁶↓S³⁷, increases BRET between PAR₂ and Rab5a and decreases BRET between PAR₂ and KRas, consistent with endocytosis. The biased agonists cathepsin-S and elastase, which respectively cleave PAR₂ at E⁵⁶↓T⁵⁷ and A⁶⁶↓S⁶⁷↓V⁶⁸, did not induce PAR₂ endocytosis. Förster resonance energy transfer (FRET) biosensors targeted to the plasma membrane, cytosol or nucleus were used to examine the capacity of proteases to generate signals in subcellular compartments of HEK cells and rat dorsal root ganglia neurons. Trypsin (internalizing) activated cAMP and protein kinase C (PKC) at the plasma membrane and in the cytosol, and extracellular signal regulate kinase (ERK) in the nucleus and cytosol. Cathepsin-S and elastase (non-internalizing) only activated cAMP and PKC at the plasma membrane and ERK in the cytosol. Dynamin inhibitor Dyngo4a, Clathrin inhibitor Pitstop2 and dominant negative β-arrestin inhibited trypsin-evoked endocytosis of PAR₂, and prevented trypsin-evoked activation of cytosolic cAMP and PKC and nuclear ERK. These results are consistent with a role for PAR₂ endocytosis in activating cytosolic cAMP and PKC and nuclear ERK. To examine the link between PAR₂ endocytosis, compartmentalized signaling and nociceptor sensitization, perforated patch clamp recordings were made from mouse dorsal root ganglia neurons. Preincubation of neurons with trypsin, cathepsin-S or elastase induced an immediate increase in excitability, assessed by decreased rheobase and increased action potential firing that was sustained for 30 min after protease washout. Dyngo4a and Pitstop2 did not affect the initial hyperexcitability induced by any protease, but prevented the sustained excitability to trypsin, but not cathepsin-S or elastase. The effects of proteases on excitability were blocked by the PAR₂ antagonist GB88. Thus, canonical and biased proteases sensitize nociceptors by distinct mechanisms. For canonical agonists, PAR₂ endocytosis generates compartmentalized signals subcellular compartments that underlie sustained excitability of nociceptive neurons. Inhibiting endosomal signaling of PAR₂ may provide a novel method of treating protease-evoked pain.

In one aspect the present invention provides a method for the treatment of protease-evoked pain comprising administering to a subject in need thereof a compound that inhibits endosomal signaling of PAR₂.

In one aspect, the compound that inhibits endosomal PAR₂ signaling is a compound that inhibits endocytosis of the activated receptor.

It will appreciated that the compound that inhibits endocytosis of PAR₂ may act at any site or at multiple sites in the pathway between phosphorylation of the intracellular C-terminus of the activated PAR₂ and subsequent dynamin- and clathrin-dependent endocytosis of the receptor to early endosomes.

In one embodiment, inhibition of endocytosis of PAR₂ may be achieved by administering to a subject in need thereof a β-arrestin inhibitor. As used herein, the term “β-arrestin inhibitor” denotes a compound that inhibits the interaction between β-arrestin and the intracellular C-terminus of the activated PAR₂.

It is envisaged that in one embodiment, inhibition of the interaction between βarrs and the intracellular C-terminus of the activated PAR₂ may be achieved by administering to a subject a J-arrestin inhibitor that competes with phosphorylation sites on the intracellular C-terminus of activated PAR₂ by providing an alternative site for GPCR kinase-2 (GRK2) phosphorylation, thereby reducing or ameliorating the binding of βarrs to the intracellular C-terminus of activated PAR₂ and subsequent endocytosis of the receptor.

In another embodiment it is envisaged that inhibition of the interaction between βarrs and the intracellular C-terminus of the activated PAR₂ may be achieved by administering to a subject a β-arrestin inhibitor that inhibits GPCR kinase-2 (GRK2) phosphorylation of the intracellular C-terminus of the activated PAR₂. In one embodiment it is envisaged that the β-arrestin inhibitor that inhibits GPCR kinase-2 (GRK2) phosphorylation of the intracellular C-terminus of activated PAR₂ interacts directly with GRK2, for example, by binding at the central catalytic domain of GRK2 responsible for receptor phosphorylation. In another embodiment, it is envisaged that the β-arrestin inhibitor may bind allosterically to GRK2, for example, to prevent recognition of phosphorylation sites on the intracellular C-terminus of the activated PAR₂. In yet another embodiment it is envisaged that the 3-arrestin inhibitor will bind at or near phosphorylation sites within the intracellular C-terminus of PAR₂ thereby preventing recognition and phosphorylation by GRK2. It is also envisaged that the compound that inhibits interaction between βarrs and the intracellular C-terminus of the activated PAR₂ may act by directly interacting with β-arrestin, inhibiting it from binding to phosphorylated sites on the intracellular C-terminus of PAR₂.

In another embodiment, inhibition of endocytosis of PAR₂ may be achieved by administering to a subject in need thereof a clathrin inhibitor to inhibit clathrin-dependent endocytosis of the activated PAR₂. It will be appreciated that the clathrin inhibitor may be any compound that inhibits clathrin-dependent endocytosis of the activated PAR₂, for example, a compound that selectively inhibits ligand association with the terminal domain of clathrin. Several clathrin inhibitors are known in the art such as Pitstop 1™ and Pitstop 2™.

In another embodiment, inhibition of endocytosis of PAR₂ may be achieved by administering to a subject in need thereof a dynamin inhibitor to inhibit dynamin- or clathrin-dependent endocytosis of the activated PAR₂. It will be appreciated that the dynamin inhibitor may be any compound that inhibits dynamin and subsequent endocytosis of the activated PAR₂, for example, a compound that inhibits L-phosphatidylserine liposome-stimulated helical dynamin, wherein dynamin is induced to form a helix around liposomes, a compound that inhibits Grb2-stimulated dynamin, or a compound that inhibits self-assembly of dynamin into single rings. Several dynamin inhibitors are known in the art such as Dyngo 4a and Dynole 2-24.

In another embodiment, inhibition of endocytosis of PAR₂ may be achieved by administering to a subject in need thereof a dominant negative β-arrestin which function by competing with endogenous arrestins for binding to clathrin and thereby inhibit clathrin-dependent endocytosis of the activated PAR₂.

In another aspect, the compound that inhibits endosomal PAR₂ signaling is a compound that targets and inhibits endosomal PAR₂ signaling.

In one embodiment, the compound that inhibits endosomal PAR₂ signaling is a tripartite compound of formula (I):

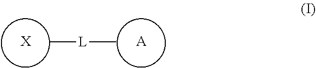

wherein
A is a lipid anchor that promotes insertion of the compound into a plasma membrane;
L is a linker moiety of 1 nm to 50 nm in length; and
X is an inhibitor of endosomal PAR₂ signaling;
wherein the lipid anchor partitions into lipid membranes that are insoluble in non-ionic detergent at 4° C.; or
a pharmaceutically acceptable salt thereof.

The term “tripartite compound” as herein used refers to compounds comprising an endosomal GPCR modulator covalently bound to a linker group, the linker group being covalently bound to a lipid anchor capable of anchoring the compound of formula (I) to the lipid bilayer of a cell membrane and ultimately, to the membrane of an early endosome.

The term “lipid anchor” as herein used denotes moieties that are capable of partitioning into lipid membranes and thereby anchoring the compound of formula (I) into the lipid membrane. The partition into the lipid membrane may occur directly from the extracellular or vesicular luminal space or may occur laterally from the lipid bilayer. The lipid anchor may be characterized by its ability to partition into lipid membranes whereby said lipid membranes are characterized by insolubility in non-ionic detergents at 4° C. Examples of suitable lipid anchors include, but are not limited to cholesterol, cholestanol, sphingolipid, GPI-anchor or saturated fatty acid derivatives. Many such lipid anchors have been described in the art, for example, in WO2005/097199, the entirety of which is incorporated herein by reference.

In one embodiment the lipid anchor is a moiety selected from formulae (II) or (III):

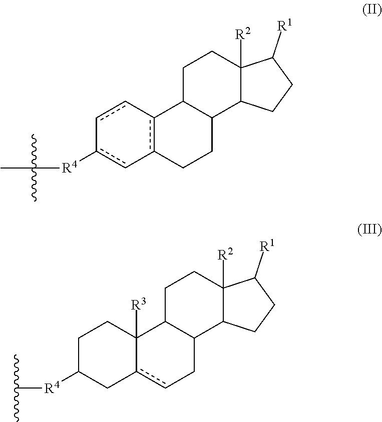

wherein
R¹ is an optionally substituted C₁₋₁₂ alkyl group;
R² and R³ are independently H or C₁₋₃alkyl;
R⁴ is —CH₂—, —O—, —NH—, —S—, —NH(CH₂)ₐOPO₃⁻—, —NH(CH₂)ₐSO₂CF₂—, —NH(CH₂)ₐSO₂NH—, —NHCONH—, —NHC(O)O—, —NHCH(CONH₂)(CH₂)_{b}C(O)O—, —NHCH(COOH)(CH₂)_{b}C(O)O—, —NHCH(CONH₂)(CH₂)_{b}CONH—, —NHCH(COOH)(CH₂)_{b}CONH—, —NHCH(CONH)(CH₂)₄NH((CO)CH₂O)ₑ— or —NHCH(COOH)(CH₂)₄NH((CO)CH₂O)ₑ—;
a is an integer from 2 to 3;
b is an integer from 1 to 2;
e is an integer from 0 to 1; and [symbol]
represents a single or double bond.

In other embodiments the lipid anchor is a moiety selected from formulae (VI), (VII), (VIII) or (IX):

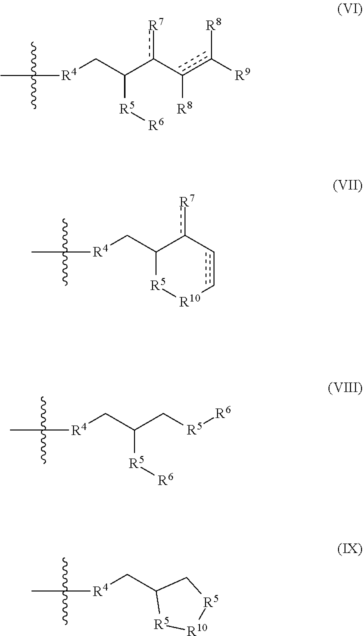

wherein
R⁴ is as described above; [symbol]
represents a single or double bond; [symbol]
represents a single, double or triple bond;
each occurrence of R⁵ is independently —NH—, —O—, —S—, —OC(O)—, —NHC(O)—, —NHCONH—, —NHC(O)O— or —NHS(O₂)—;
each occurrence of R⁶ is independently a C₁₄₋₃₀ alkyl group optionally substituted by fluorine, preferably 1 to 4 fluorine atoms;
each occurrence of R⁷ is independently NH₂, NHCH₃, OH, H, halogen or O, provided that when R⁷ is NH₂, NHCH₃, OH, H or halogen then [symbol] is a single bond and when R⁷ is O then [symbol] is a double bond;
each occurrence of R⁸ is independently H, OH or is absent when [symbol] represents a triple bond;
R⁹ is a C₁₀₋₃₀ alkyl group optionally substituted by fluorine, preferably 1 to 4 fluorine atoms; and
each occurrence of R¹⁰ is independently a C₂₄₋₄₀ alkylene group, a C₂₄₋₄₀ alkenylene group or a C₂₄₋₄₀ alkynylene group optionally substituted by fluorine, preferably 1 to 4 fluorine atoms.

In further embodiments, the lipid anchor is a moiety selected from formulae (X) or (XI):

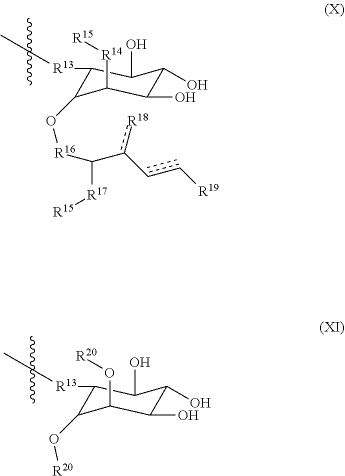

wherein [symbol]
represents a single or double bond; [symbol]
represents a single, double or triple bond;
each occurrence of R¹³ is independently —O— or —CO(CH₂)ₐ(CO)_{b}O—, wherein a is an integer from 1 to 3 and b is an integer from 0 to 1;

### R¹⁴ is —O— or —OC(O)—;

each occurrence of R¹⁵ is independently selected from a C₁₆₋₃₀ alkyl group optionally substituted with fluorine, preferably 1 to 4 fluorine atoms;
R¹⁶ is —PO₃—CH₂—, —SO₃CH₂—, —CH₂—, —CO₂CH₂— or a direct bond;
R¹⁷ is —NH—, —O—, —S—, —OC(O)—, —NHC(O)—, —NHCONH—, —NHC(O)O— or —NHS(O₂)—;
R¹⁸ is NH₂, NHCH₃, OH, H, halogen or O;
R¹⁹ is a C₁₆₋₃₀ alkyl group optionally substituted with fluorine, preferably 1 to 4 fluorine atoms; and
each R²⁰ is a C(O)C₁₃₋₂₅alkyl group optionally substituted with a group of the following formulae:

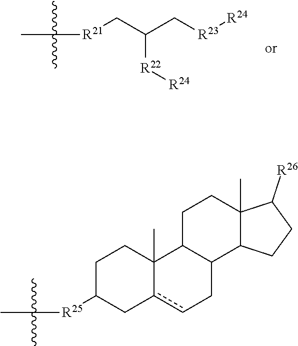

wherein [symbol]
is a single or double bond;
R²¹ is —PO₃—CH₂—, —SO₃CH₂—, —CH₂—, —CO₂CH₂— or a direct bond;
R²² is —NH—, —O—, —S—, —OC(O)—, —NHC(O)—, —NHCONH—, —NHC(O)O— or —NHS(O₂)—;

### R²³ is —O— or —OC(O)—;

each occurrence of R²⁴ is independently selected from a C₁₆₋₃₀ alkyl group optionally substituted with fluorine, preferably 1 to 4 fluorine atoms;
R²⁵ is —CO(CH₂)ₐ(CO)_{b}O— or —CO(CH₂)ₐ(CO)_{b}NH—, wherein a is an integer from 1 to 3 and b is an integer from 0 to 1; and
R²⁶ is a C₄₋₂₀ alkyl group optionally substituted with fluorine, preferably 1 to 4 fluorine atoms.

In further embodiments the lipid anchor is a moiety selected from formulae (XII), (XIII), (XIV) or (XV):

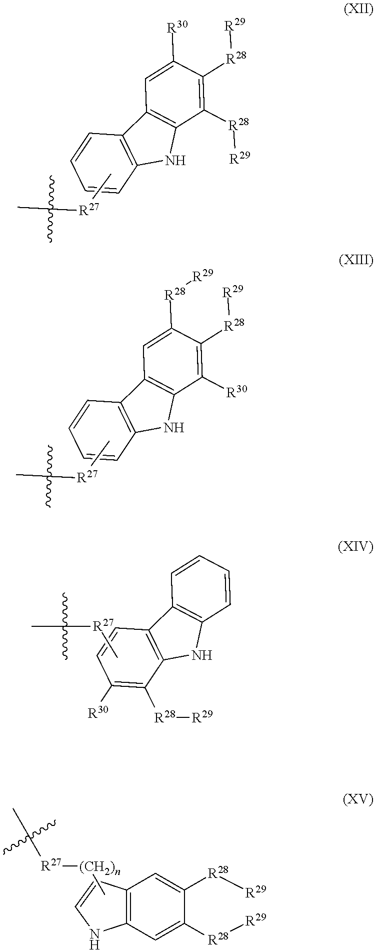

wherein
each occurrence of R²⁷ is independently selected from —NH—, —O—, —NH(CH₂)_{c}OPO₃—, —NH(CH₂)_{c}SO₂NH—, —NHCONH—, —NHC(O)O—, —CO(CH₂)_{b}(CO)ₐNH—, —CO(CH₂)_{b}(CO)ₐO—, —CO(CH₂)_{b}S—, —CO(CH₂)_{b}OPO₃—, —CO(CH₂)_{b}SO₂NH—, —CO(CH₂)_{b}NHCONH—, —CO(CH₂)_{b}OCONH—, —CO(CH₂)_{b}OSO₃—, or —CO(CH₂)_{b}NHC(O)O—, wherein a is an integer from 0 to 1, b is an integer from 1 to 3 and c is an integer from 2 to 3;
each occurrence of R²⁸ is independently —CH₂— or —O—;
each occurrence of R²⁹ is independently selected from H or a C₁₆₋₃₀ alkyl group optionally substituted by fluorine, preferably 1 to 4 fluorine atoms;
each occurrence of R³¹ is independently selected from H, or a C₁₋₁₅ alkyl group, optionally substituted by fluorine, preferably 1 to 4 fluorine atoms, or a C₁₋₁₅ alkoxy group optionally substituted by fluorine, preferably 1 to 4 fluorine atoms; and
n is an integer from 1 to 2.

The term “linker” as herein used relates to the part of the compound that links the modulator of an endosomal GPCR to the lipid anchor. It will be understood that the linker should be selected such that it does not compete with the modulator of an endosomal GPCR at the ligand binding site. Nor should the linker partition into the lipid membrane. The linker group should be of a length of between 1 nm to 50 nm in order to allow the modulator of an endosomal GPCR to interact with the receptor when the lipid anchor is anchored in the endosome membrane. In one embodiment, the linker group will comprise one or more polyethelene glycol units. In another embodiment it is envisaged that the linker, or subunits of the linker, may be amino acid residues, derivatised or functionalised amino acid residues, polyethers, ureas, carbamates, sulphonamides or other subunits that provide adequate distance between the modulator of an endosomal GPCR and the lipid anchor without interfering in the function of either group.

In one embodiment, the linker is represented by a moiety of the formula (IV):

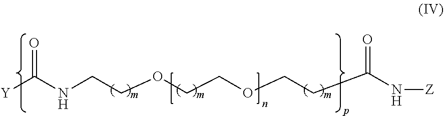

wherein
Z is the attachment group between the linker and the lipid anchor and is —C₁-C₁₀alkyl-, —C₂-C₁₀alkenyl-, —C₂-C₁₀alkynyl-, —C₁-C₁₀alkylC(O)—, —C₂-C₁₀alkenylC(O)— or —C₂-C₁₀alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
Y is the attachment group between the linker and the modulator of an endosomal GPCR and is —O—, —NH—, —S—, —C(O)—, —C(O)NH—, —C(O)O— or —C(O)S—; or
Y, together with the adjacent amido group is an amino acid selected from aspartic acid, glutamic acid, asparagine, glutamine, histidine, cysteine, lysine, arginine, serine or threonine; wherein the amino acid is attached to the modulator of an endosomal GPCR via its side-chain functional group;
m is 1 or 2;
n is from 1 to 20; and
p is from 1 to 8.

In another embodiment, the linker is represented by a moiety of the formula (XX):

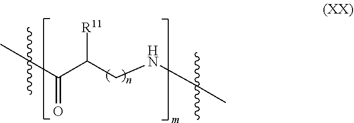

wherein
each occurrence of R¹¹ is independently any side chain of a naturally occurring, derivatised or functionalised amino acid residue;
m is an integer from 3 to 80; and
n is an integer from 0 to 1.

In other embodiments, the linker is represented by a moiety of the formula (XXI):

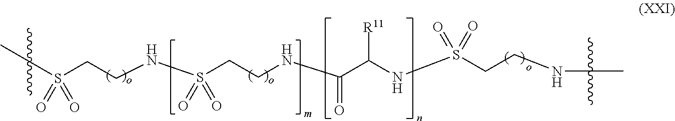

wherein
m is an integer from 0 to 40;
n is an integer from 0 to 1;
each occurrence of o is independently an integer from 1 to 5;
each occurrence of R¹¹ is independently any side chain of a naturally occurring, derivatised or functionalised amino acid residue; and
wherein the SO₂ terminus is bound to the lipid anchor and the N-terminus is bound to the modulator of an endosomal GPCR.

In a further embodiment, the linker is represented by a moiety of the formula (XXII):

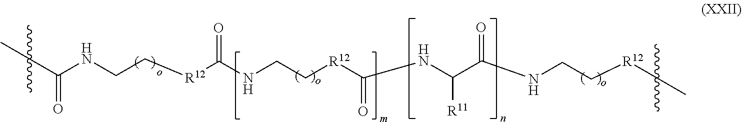

wherein
m is an integer from 0 to 40;
n is an integer from 0 to 1;
each occurrence of o is independently an integer from 1 to 5;
each R¹² is independently NH or O;
each occurrence of R¹¹ is independently any side chain of a naturally occurring, derivatised or functionalised amino acid residue; and
wherein the C(O)-terminus is bound to the lipid anchor and the R¹²-terminus is bound to the modulator of an endosomal GPCR.

A number of suitable linker moieties have been described WO2005/097199, the entirety of which is incorporated herein by reference.

The term “inhibitor of endosomal PAR₂ signaling” as herein used refers to antagonists or inhibitors of PAR₂ that has been endocytosed into endosomes. The inhibitor of endosomal PAR₂ signaling may be in any form including, but not limited to, an organic molecule, a polypeptide sequence, a hormone, a protein fragment or a derivative of any of these.

The term “endosomal PAR₂ signaling” as herein used refers to the signal transduced by activated PAR₂ that has been endocytosed into an endosome, preferably an early endosome.

In one embodiment endosomal PAR₂ signaling will be signaling that is first transduced at the plasma membrane and is maintained when the receptor is endocytosed into early endosomes.

In another embodiment, the endosomal PAR₂ signaling will be signaling that requires receptor endocytosis and/or occurs exclusively on endosomal membranes, for example, β-arrestin mediated signaling. It is believed that βarrs interact with agonist-occupied G protein-coupled receptor kinase (GRK)-phosphorylated GPCRs at the cell surface and promote the transfer of ligand-bound receptor from the cell surface to early endosomes via dynamin- and clathrin-dependent endocytosis. It has recently been discovered that this pathway can mediate a second series of endosomal GPCR signaling that is distinct from G protein-dependent signaling at the plasma membrane. It is believed that the importance of this mechanism depends on the affinity with which GPCRs interact with βarrs, which varies depending on the extent of GPCR phosphorylation by GRKs. “Class A” GPCRs (e.g., β₂AR, α_{1b}AR) have few phosphorylation sites, and transiently interact with βarr1 and βarr2, mostly at the plasma membrane, with a higher affinity for βarr2. “Class B” GPCRs (e.g., AT_{1A}R, NK₁R, PAR₂) are phosphorylated at multiple sites and interact with both βarr1 and 2 with high affinity for prolonged periods at plasma and endosomal membranes. “Class C” GPCRs (e.g., bradykinin B₂ receptor) internalize with βarrs into endosomes followed by rapid dissociation of βarr upon agonist removal.

It is believed that the extent of βarr-induced MAPK signaling depends on the affinity of the receptor for βarrs, which depends on the receptor structure and on which of the seven mammalian GRKs phosphorylate the receptor. Thus, activation of AT_{1A}R and V₂R causes greater phosphorylation of βarr-bound ERK1/2 than activation of α_{1b}AR and β₂AR, suggesting that the class B receptors signal more robustly through this pathway. As mentioned above, PAR₂ is a Class B GPCR.

In some preferred embodiments of the invention, and with reference to the general formula (I), one or more of the following embodiments apply:

a) A is a lipid anchor selected from cholesterol, cholestanol, sphingolipid, a GPI-anchor or a saturated fatty acid derivative.
b) A is a lipid anchor selected from moieties of formulae (II), (III), (VII), (VIII), (IX), (X), (XI), (XII), (XIII) and (IX).
c) A is a lipid anchor selected from moieties of formulae (II) or (III).
d) L is a linker moiety comprising one or more subunits, the subunits comprising polyethelene glycol units, amino acid residues, derivatised or functionalised amino acid residues, polyethers, ureas, carbamates and/or sulphonamides.
e) L is a linker moiety represented by formulae (IV), (XX), (XXI) or (XXII).
f) L is a linker moiety represented by formula (IV).

In a preferred embodiment A is a lipid anchor represented by formulae (II) or (III).

Accordingly, in one embodiment, the present invention provides tripartite compounds of the formula (I) represented by formula (Ia):

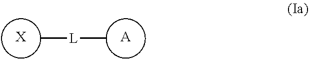

wherein
A is a lipid anchor that promotes insertion of the compound into a plasma membrane represented by formulae (II) or (III):

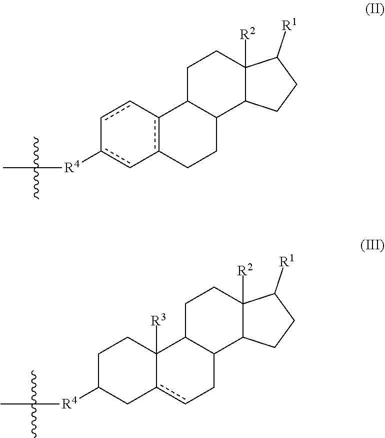

wherein
R¹ is an optionally substituted C₁₋₁₂ alkyl group;
R² and R³ are independently H or C₁₋₃alkyl;

### R⁴ is C, O, NH or S; and

[symbol] represents a single or double bond;
L is a linker group of 1 nm to 50 nm in length; and
X is an inhibitor of endosomal PAR₂ signaling; or
pharmaceutically acceptable salts thereof.

In another preferred embodiment A is a lipid anchor represented by formulae (II) or (III) and L is a linker represented by formula (IV).

Accordingly, in another embodiment, the present invention provides tripartite compounds of the formula (I) represented by formula (Ib):

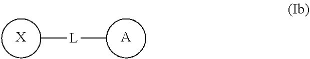

wherein
A is a lipid anchor that promotes insertion of the compound into a plasma membrane represented by formulae (II) or (III):

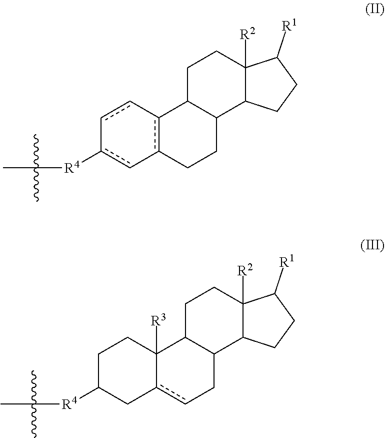

wherein
R¹ is an optionally substituted C₁₋₁₂ alkyl group;
R² and R³ are independently H or C₁₋₃alkyl;

### R⁴ is C, O, NH or S;

[symbol] represents a single or double bond;
L is represented by the formula (IV):

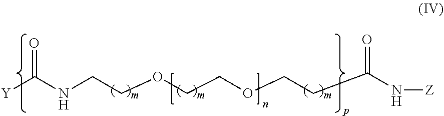

wherein
Z is the attachment group between the linker and the lipid anchor and is —C₁-C₁₀alkyl-, —C₂-C₁₀alkenyl-, —C₂-C₁₀alkynyl-, —C₁-C₁₀alkylC(O)—, —C₂-C₁₀alkenylC(O)— or —C₂-C₁₀alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
Y is the attachment group between the linker and the modulator of an endosomal GPCR and is —O—, —NH—, —S—, —C(O)—, —C(O)NH—, —C(O)O— or —C(O)S—; or Y, together with the adjacent amido group is an amino acid selected from aspartic acid, glutamic acid, asparagine, glutamine, histidine, cysteine, lysine, arginine, serine or threonine; wherein the amino acid is attached to the modulator of an endosomal GPCR via its side-chain functional group;
m is 1 or 2;
n is from 1 to 20;
p is from 1 to 8; and
X is an inhibitor of endosomal PAR₂ signaling; or
pharmaceutically acceptable salts thereof.

In one embodiment, the compound of formula (I) has the structure:

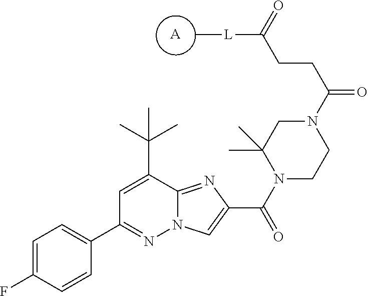

wherein L and A are as defined herein, or a pharmaceutically acceptable salt thereof.

In another aspect, the present invention provides the compound:

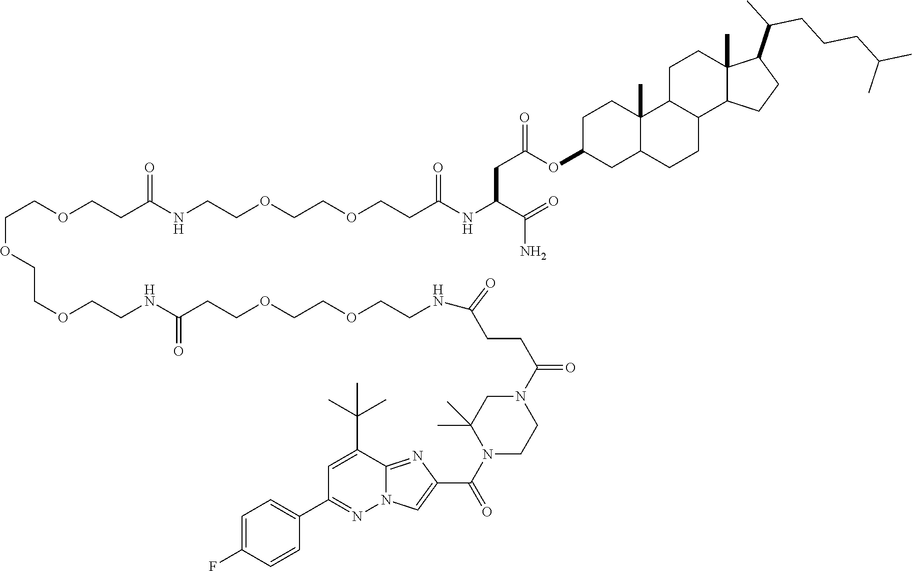

or a pharmaceutically acceptable salt thereof.

In one aspect, the present invention provides a method for the treatment of protease-evoked pain comprising administering to a subject in need thereof an effective amount of a compound of formula (I) as herein defined.

In another aspect, the present invention provides use of a compound of formula (I) as herein defined in the manufacture of a medicament for the treatment of protease-evoked pain.

In a further aspect, the present invention provides a compound of formula (I) as herein defined for use in the treatment of protease-evoked pain.

In yet a further aspect, the present invention provides a method for the treatment of protease-evoked pain comprising administering to a subject in need thereof a combination comprising one or more compounds that inhibit endocytosis of the receptor and one or more compounds that target and inhibit endosomal PAR₂ signaling.

Within the context of the present invention, the term “pain” includes chronic inflammatory pain (e.g. pain associated with rheumatoid arthritis, osteoarthritis, rheumatoid spondylitis, gouty arthritis and juvenile arthritis); musculoskeletal pain, lower back and neck pain, sprains and strains, neuropathic pain, sympathetically maintained pain, myositis, pain associated with cancer and fibromyalgia, pain associated with migraine, pain associated with cluster and chronic daily headache, pain associated with influenza or other viral infections such as the common cold, rheumatic fever, pain associated with functional bowel disorders such as non-ulcer dyspepsia, non-cardiac chest pain and irritable bowel syndrome, pain associated with myocardial ischemia, post operative pain, headache, toothache, dysmenorrhea, neuralgia, fibromyalgia syndrome, complex regional pain syndrome (CRPS types I and II), neuropathic pain syndromes (including diabetic neuropathy, chemoterapeutically induced neuropathic pain, sciatica, non-specific lower back pain, multiple sclerosis pain, HIV-related neuropathy, post-herpetic neuralgia, trigeminal neuralgia) and pain resulting from physical trauma, amputation, cancer, toxins or chronic inflammatory conditions. In a preferred embodiment the pain is somatic pain or visceral pain.

General strategies for synthesising the compounds of the invention are outlined below. Synthesis of cholesteryl glycolic acid, 3-cholesterylamine, and cholesteryl glycine are described in the literature (Hussey, S. L. et al., J. Am. Chem. Soc. 2001, 123, 12712-12713; Hussey, S. L. et al., Org. Lett. 2002, 4, 415-418; Martin, S. E. et al., Bioconjugate Chem. 2003, 14, 67-74). Lipid anchors of the formula (III) having an amide, sulfonamide, urea or carbamate functional group at position 3 of the steroid structure can be prepared from 3-cholesterylatnine, for example, 3-cholesterylamine can be reacted with succinic anhydride in the presence of DMAP to afford the corresponding succinyl substituted compound. The corresponding sulfonamide can be obtained by reaction of 3-cholesterylamine with chlorosulfonylacetic acid, which can be prepared as described in the literature (Hinman, R. L. and Locatell, L. J. Am. Chem. Soc. 1959, 81, 5655-5658). The corresponding urea or carbamate can be prepared according to literature procedures via the corresponding isocyanate (Knolker, H.-J. T. et al., Angew. Chem. Int. Ed. 1995, 34, 2497; Knolker, H.-J. et al., Synlett 1996, 502; Knolker, H.-J. and. Braxmeier, T. Tetrahedron Lett. 1996, 37, 5861). Intermediates of compound (III) having a phosphate or carboxymethylated phosphate at position 3 of the steroid structure can be prepared as described in the literature (Golebriewski, Keyes, Cushman, Bioorg. Med. Chem. 1996, 4, 1637-1648; Cusinato, Habeler, et al., J. Lipid Res. 1998, 39, 1844-1851; Himber, Missano, et al., J. Lipid Res. 1995, 36, 1567-1585). Lipid anchors of the formula (III) having a thiol at position 3 of the steroid structure can be prepared as described in the literature (J. G. Parkes, J. G. et al., Biochim. Biophys. Acta 1982, 691, 24-29), the corresponding carboxymethylated thiols are obtainable by simple alkylation as described for the corresponding amines and alcohols. Lipid anchors of the formula (III) having a difluoromethylenesulfone derivative at position 3 of the steroid structure can be prepared as described in the literature (Lapiene, J. et al., Bioorg. Med. Chem. Lett. 2004, 14, 151-155). Introduction of various side chains at position 17 of lipid anchors of the formula (III) can be achieved by use of literature protocols starting from dehydroisoandrosterone or pregnenolone (Bergmann, E. D. et al., J. Am. Chem. Soc. 1959, 81, 1239-1243 and references therein). Lipid anchors of the formula (III) which are derived from cholestane are obtainable from the corresponding precursors which are derived from cholesterol by reduction of the 5,6-double bond using literature protocols, e.g. hydrogenation in the presence of various transition metal catalysts.

Lipid anchors of the formula (II) having an oxygen derived substituent at position 3 are prepared in a similar manner as described for the lipid anchors of the formula (III) starting from estrone. Lipid anchors of the formula (II) having nitrogen derived substitution at position 3 can be prepared in a similar manner as described for lipid anchors of the formula (III) starting from 3-amino estrone, which can be prepared as described in the literature (Zhang, X. and Sui, Z. Tetrahedron Lett. 2003, 44, 3071-3073; Woo, L. W. L. et al., Steroid Biochem. Molec. Biol. 1996, 57, 79-88). Lipid anchors of the formula (II) having a sulfur derived substituent at position 3 can be prepared in a similar manner as described for lipid anchors of the formula (III) starting from 3-thioestrone, which can be prepared as described in the literature (Woo, L. W. L. et al., J. Steroid Biochem. Molec. Biol. 1996, 57, 79-88). Introduction of various side chains at position 17 of the estrone structure can be achieved by a Wittig approach, followed by hydrogenation of the resulting double bond as described in the literature (Peters, R. H. et al., J. Org. Chem. 1966, 31, 24-26). Further manipulations within the side chain (e.g. double bond constructions, cycloalkyl decorations) can be achieved by standard protocols (Suzuki-couplings, etc.).

Lipid anchors of the formula (VI) belonging to the class of ceramides, dehydroceramides and dihydroceramides with different hydrocarbon groups are obtainable as outlined in the literature (A. H. Merrill, Jr., Y. A. Hannun (Eds.), Methods in Enzymology, Vol. 311, Academic Press, 1999; Koskinen, P. M and Koskinen, A. M. P. Synthesis 1998, 1075). In particular, sphingosine base can be used as key intermediate for all lipid anchors of the formula (VI) having oxygen derived substitution at position 1 of the sphingosine backbone. The corresponding amino derivatives are obtainable by substitution of the sulfonates, which can be prepared from the alcohols according to known protocols. Alkylation and acylation of 1-amino or 1-hydroxy derivatives can be achieved by reaction with bromo acetic acid and succinic anhydride, respectively. The thioacetylated derivative can be prepared by substitution of a sulfonate with mercapto acetic acid. Phosphate and sulfate derivatives are obtainable as described in the literature (A. H. Merrill, Jr., YA A. Hannun (Eds.), Methods in Enzymology, Vol. 311, Academic Press, 1999; Koskinen, P. M. and Koskinen, A. M. P. Synthesis 1998, 1075). Acylation, sulfonylation, urea and carbamate formation can be achieved by standard procedures. Lipid anchors of the formula (VI) wherein R⁵ is an amino or amino derived function can be prepared starting from sphingosine base, which is available as published by Koskinen (Koskinen, P. M. and Koskinen, A. M. P. Synthesis 1998, 1075), using standard protocols. The corresponding 2-oxygen substituted sphingolipids can be obtained by a strategy published by Yamanoi (Yamanoi, T. et al., Chem. Lett. 1989, 335). Lipid anchors of the formula (VI), wherein both R⁸ represent a hydroxy group, are obtainable by bishydroxylation of the corresponding alkene using known protocols. The corresponding monohydroxy derivatives can be prepared as described in the literature (Howell, A. R. and Ndakala, A. J. Curr. Org. Chem. 2002, 6, 365-391). Modification of substituents R⁶ and R⁹ in lipid anchors of the formula (VI) can be achieved by protocols and strategies outlined in various review articles (Harwood, H. J. Chem. Rev. 1962, 62, 99-154; Gensler, W. J. Chem. Rev. 1957, 57, 191-280).

Lipid anchors of the formula (VII) are obtainable by protocols described in the literature (Müller, S. et al., J. Prakt. Chem. 2000, 342, 779) and by combinations thereof with protocols described for the preparation of lipid anchors of the formula (VII).

Lipid anchors of the formula (VIII), wherein R⁴ and R⁵ are oxygen derived substituents, can be prepared starting from commercially available (R)-(−)-2,2-dimethyl-1,3-dioxolane-4-methanol as outlined by Fraser-Reid (Schlueter, U. Lu, J. and Fraser-Reid, B. Org. Lett. 2003, 5, 255-257). Variation of substituents R⁶ in compounds of formula (VIII) can be achieved by protocols and strategies outlined in various review articles (Harwood, H. J. Chem. Rev. 1962, 62, 99-154; Gensler, W. J. Chem. Rev. 1957, 57, 191-280). Lipid anchors of the formula (VIII), wherein R⁴ and R⁵ are nitrogen derived substituents, are obtainable either starting from the corresponding oxygen substituted systems by nucleophilic replacement of the corresponding sulfonates and further modifications as outlined above, or starting from 1,2,3-triaminopropane which is obtainable as described in the literature (Henrick, K. et al., J Chem. Soc. Dalton Trans. 1982, 225-227).

Lipid anchors of the formula (IX) are obtainable in a similar fashion as lipid anchors of the formula (VII) or alternatively by ring closing metathesis of co-ethenylated intermediates of lipid anchors of the formula (VIII).

Lipid anchors of the formulae (X) and (XI) are obtainable by synthetic strategies described in the literature (Xue, J. and Guo, Z. Bioorg. Med. Chem. Lett. 2002, 12, 2015-2018; Xue, J. and Guo, Z. J. Am. Chem. Soc. 2003, 16334-16339; Xue, J. et al., J. Org. Chem. 2003, 68, 4020-4029; Shao, N., Xue, J. and Guo, Z. Angew. Chem. Int. Ed. 2004, 43, 1569-1573) and by combinations thereof with methods described above for the preparation of lipid anchors of the formulae (VI) and (VIII).

Lipid anchors of the formulae (XII), (XIII) and (XIV) are obtainable by total synthesis following synthetic strategies described in the literature (Knolker, H.-J. Chem. Soc. Rev. 1999, 28, 151-157; Knolker, H.-J. and Reddy, K. R. Chem. Rev. 2002, 102, 4303-4427; Knolker, H.-J. and Knoll, J. Chem. Commun. 2003, 1170-1171; Knolker, H.-J. Curr. Org. Synthesis 2004, 1).

Lipid anchors of the formula (XV) can be prepared by Nenitzescu-type indole synthesis starting from 4-methoxy-3-methylbenzaldehyde to afford 6-methoxy-5-methylindole. Ether cleavage, triflate formation and Sonogashira coupling leads to the corresponding 6-alkynyl substituted 5-methylindole. Nilsmeier formylation and subsequent nitromethane addition yields the 3-nitro vinyl substituted indole derivative which is subjected to a global hydrogenation resulting in the formation of the 6-alkyl substituted 5-methyltryptamine. Acylation of the amino group using succinyl anhydride completes the preparation.

Known solid or solution phase techniques may be used in the synthesis of the peptides of the present invention, such as coupling of the N- or C-terminus to a solid support (typically a resin) followed by step-wise synthesis of the linear peptide. Protecting group chemistries for the protection of amino acid residues, including side chains, are well known in the art and may be found, for example, in: Theodora W. Greene and Peter G. M. Wuts, Protecting Groups in Organic Synthesis (Third Edition, John Wiley & Sons, Inc, 1999), the entire contents of which is incorporated herein by reference.

Methods for the preparation of compounds as described herein will be apparent to those skilled in the art and will comprise the steps of a) defining the distance between (a) phosphoryl head group(s) or an equivalent head group of the lipid anchor and a binding and/or interaction site of the modulator of the inhibitor of endosomal PAR₂ signaling; b) selecting a linker which is capable of spanning the distance as defined in (a); and c) bonding the lipid anchor and the inhibitor of endosomal PAR₂ signaling by the linker as selected in (b).

Corresponding working examples for such a method are given herein. The person skilled in the art is in a position to deduce relevant binding sites or interactions sites of a given or potential inhibitor of endosomal PAR₂ signaling and, accordingly, to determine the distance between (a) phosphoryl head group(s) or an equivalent head group of the lipid anchor and a binding and/or interaction site of the inhibitor of endosomal PAR₂ signaling. Such methods comprise, but are not limited to molecular modelling, in vitro and/or molecular-interaction or binding assays (e.g. yeast two or three hybrid systems, peptide spotting, overlay assays, phage display, bacterial displays, ribosome displays), atomic force microscopy as well as spectroscopic methods and X-ray crystallography. Furthermore, methods such as site-directed mutagenesis may be employed to verify deduced interaction sites of a given inhibitor of endosomal PAR₂ signaling or of a candidate inhibitor of endosomal PAR₂ signaling and its corresponding target.

The skilled addressee will understand that the selection of a linker comprises the selection of linkers known in the art as well as the generation and use of novel linkers, for example, by molecular modelling and corresponding synthesis or further methods known in the art. The term “spanning” as used herein with reference to step b) refers to the length of the linker selected to place the inhibitor of endosomal PAR₂ signaling at the correct locus on the a receptor when the lipid anchor forms part of the lipid layer of the endosome.

The skilled addressee is readily in the position to deduce, verify and/or evaluate the lipophilicity of a given tripartite compound as well as of the individual moiety as described herein. Corresponding test assays to determine endosomal GPCR targeting are provided herein in the examples.

The skilled addressee will understand that the purpose of the linker moiety is to connect the lipid anchor to the inhibitor of endosomal PAR₂ signaling in order to allow the inhibitor of endosomal PAR₂ signaling to interact with PAR₂ when the lipid anchor is anchored in the endosome membrane. The lipid anchor and the linker will contain functional groups allowing for the two to be covalently bonded. The nature of the functional group of the lipid anchor is in no way limited and may include, for example, an amine group that forms an amide bond with the linker, or a hydroxyl or carboxylic acid group that forms and ether or ester bond with the linker.

Similarly, the skilled addressee will understand that selection of the functional group at the end of the linker that connects with the inhibitor of endosomal PAR₂ signaling will be dictated primarily by any available functional groups on the inhibitor of endosomal PAR₂ signaling of choice. For example, if the inhibitor of endosomal PAR₂ signaling comprises a free amine or carboxylic acid group, it is envisaged that the functional group of the linker will comprise a complementary carboxylic acid or amine to form an amide bond.

It will be understood that the compounds of the present invention may exist in one or more stereoisomeric forms (e.g. diastereomers). The present invention includes within its scope all of these stereoisomeric forms either isolated (in, for example, enantiomeric isolation), or in combination (including racemic mixtures and diastereomic mixtures). The present invention contemplates the use of amino acids in both L and D forms, including the use of amino acids independently selected from L and D forms, for example, where the peptide comprises two serine residues, each serine residue may have the same, or opposite, absolute stereochemistry. Unless stated otherwise, the amino acid is taken to be in the L-configuration.

The invention thus also relates to compounds in substantially pure stereoisomeric form with respect to the asymmetric centres of the amino acid residues, e.g., greater than about 90% de, such as about 95% to 97% de, or greater than 99% de, as well as mixtures, including racemic mixtures, thereof. Such diastereomers may be prepared by asymmetric synthesis, for example, using chiral intermediates, or mixtures may be resolved by conventional methods, e.g., chromatography, or use of a resolving agent.

Where the compounds of the present invention require purification, chromatographic techniques such as high-performance liquid chromatography (HPLC) and reverse-phase HPLC may be used. The peptides may be characterised by mass spectrometry and/or other appropriate methods.

Where the compound comprises one or more functional groups that may be protonated or deprotonated (for example at physiological pH) the compound may be prepared and/or isolated as a pharmaceutically acceptable salt. It will be appreciated that the compound may be zwitterionic at a given pH. As used herein the expression “pharmaceutically acceptable salt” refers to the salt of a given compound, wherein the salt is suitable for administration as a pharmaceutical. Such salts may be formed, for example, by the reaction of an acid or a base with an amine or a carboxylic acid group respectively.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts may be prepared from inorganic and organic bases. Corresponding counter ions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine.

Acid/base addition salts tend to be more soluble in aqueous solvents than the corresponding free acid/base forms.

The present invention also provides a pharmaceutical composition comprising a therapeutically effective amount of a compound as hereinbefore defined, or a pharmaceutically acceptable salt thereof, together with at least one pharmaceutically acceptable carrier or diluent.

The term “composition” is intended to include the formulation of an active ingredient with encapsulating material as carrier, to give a capsule in which the active ingredient (with or without other carrier) is surrounded by carriers.

While the compounds as hereinbefore described, or pharmaceutically acceptable salts thereof, may be the sole active ingredient administered to the subject, the administration of other active ingredient(s) with the compound is within the scope of the invention. In one or more embodiments it is envisaged that a combination of two or more of the compounds of the invention will be administered to the subject. It is envisaged that the compound(s) could also be administered with one or more additional therapeutic agents in combination. The combination may allow for separate, sequential or simultaneous administration of the compound(s) as hereinbefore described with the other active ingredient(s). The combination may be provided in the form of a pharmaceutical composition.

The term “combination”, as used herein refers to a composition or kit of parts where the combination partners as defined above can be dosed dependently or independently or by use of different fixed combinations with distinguished amounts of the combination partners, i.e., simultaneously or at different time points. The combination partners can then, e.g., be administered simultaneously or chronologically staggered, that is at different time points and with equal or different time intervals for any part of the kit of parts. The ratio of the total amounts of the combination partners to be administered in the combination can be varied, e.g. in order to cope with the needs of a patient sub-population to be treated or the needs of the single patient which different needs can be due to age, sex, body weight, etc. of the patients.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the active compound care should be taken to ensure that the activity of the compound is not destroyed in the process and that the compound is able to reach its site of action without being destroyed. In some circumstances it may be necessary to protect the compound by means known in the art, such as, for example, micro encapsulation. Similarly the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The compounds as hereinbefore described, or pharmaceutically acceptable salts thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the active compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the compounds of the invention in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active compound may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the compounds of the invention may be incorporated into sustained-release preparations and formulations, including those that allow specific delivery of the active peptide to specific regions of the gut.

Liquid formulations may also be administered enterally via a stomach or oesophageal tube. Enteral formulations may be prepared in the form of suppositories by mixing with appropriate bases, such as emulsifying bases or water-soluble bases. It is also possible, but not necessary, for the compounds of the present invention to be administered topically, intranasally, intravaginally, intraocularly and the like.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 2000 mg. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

As used herein, the term “effective amount” refers to an amount of compound which, when administered according to a desired dosing regimen, provides the desired therapeutic activity. Dosing may occur once, or at intervals of minutes or hours, or continuously over any one of these periods. Suitable dosages may lie within the range of about 0.1 ng per kg of body weight to 1 g per kg of body weight per dosage. A typical dosage is in the range of 1 μg to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

The terms “treatment” and “treating” as used herein cover any treatment of a condition or disease in an animal, preferably a mammal, more preferably a human, and includes treating pain mediated by endosomal PAR₂ signaling. The terms “prevention” and “preventing” as used herein cover the prevention or prophylaxis of a condition or disease in an animal, preferably a mammal, more preferably a human and includes prevention pain mediated by endosomal PAR₂ signaling.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The invention will now be described with reference to the following non-limiting examples:

### Example 1: BRET Studies of Receptor Trafficking

Bret studies of receptor trafficking were conducted to quantify PAR₂ internalisation (RIT-venus) and trafficking to early endosomes (Rab5a-venus) upon stimulation with different agonists.

### Methods

After achieving ˜50% confluency within 10 cm dishes HEK293 cells were transiently transfected with 1 μg PAR₂-RLuc8 and 4 μg of either RIT-venus or Rab5a-venus using PEI as a transfection agent. 24 hours post transfection, cells were seeded into poly-D-lysine coated 96-well isoplates. The following day cell culture media was replaced with either 1×HBSS (+0-28 g HEPES/100 ml, pH 7-40); 30 μM Dyngo4a (Dyn4a); 30 μM inactive Dyn4a control; 30 μM PitStop2 (PS2) or 30 μM inactive PS2 control. When used endocytic inhibitors were diluted in 1×HBSS+1% dimethyl sulfoxide (DMSO). Following the replacement of culture media cells were allowed to equilibrate at 37° C. for 30 minutes before the addition of colenterazine H [5 μM]. After a further 5 minute incubation BRET was assessed using a LUMIstar Omega microplate reader (BMG LabTech, Offenburg, Germany), with luminescence being read at 535 nm and 475 nm. A 4 minute baseline was obtained before the addition of vehicle (1×HBSS+/−1% DMSO) or protease, measurements were then continued for a further 30 minutes. BRET ratios were calculated using LUMIstar MARS analysis software and then corrected to baseline and vehicle treatments in Microsoft Excel 2013.

### Results

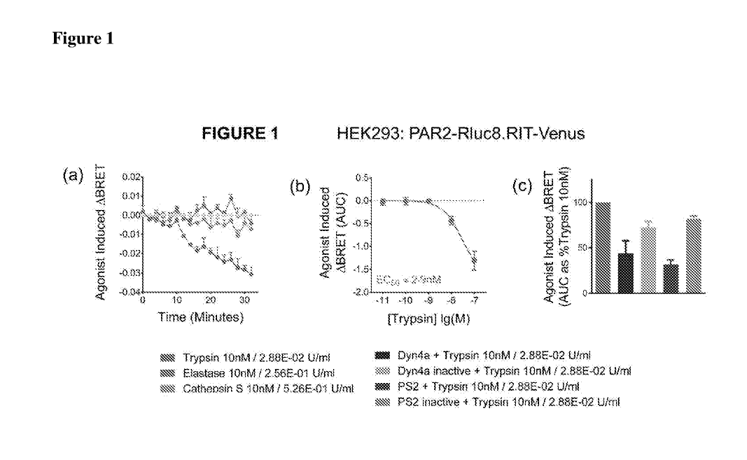

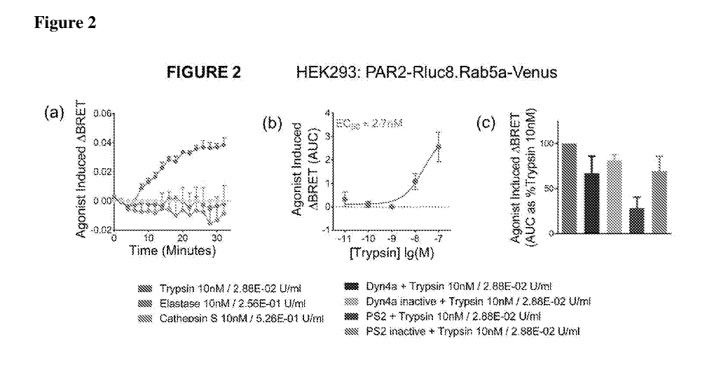

Initial trafficking experiments showed trypsin to be the only agonist to lead to internalisation of PAR₂. Stimulation of the receptor with a 10 nM concentration of trypsin resulted in a time dependent decrease in BRET with the plasma membrane marker (FIG. 1a). This coincided with an increase in BRET between the receptor and the early endosomal marker (FIG. 2a). Such responses are consistent with receptor internalisation and were not observed upon stimulating the receptor with elastase or cathepsin S. It is thus believed that elastase and cathepsin S activated PAR₂ can only signal from the plasma membrane.

The ability of trypsin to induce receptor internalisation was further explored by administering a range of concentrations to cells. It was found that trypsin induced internalisation of PAR₂ is concentration dependent (FIGS. 1b and 2b). Similar EC₅₀ concentrations resulted from assays with both markers (˜2.8 nM).

Pharmacological inhibition of clathrin by PS2 reduced the magnitude of BRET between the luciferase tagged receptor and both markers upon trypsin stimulation, consistent with inhibition of internalisation. A less pronounced inhibition of trypsin evoked internalisation was seen upon inhibition of dynamin with Dyn4a (FIGS. 1c and 2c).

### Example 2: Whole Population FRET to Assess Compartmentalised ERK Signaling

Förster Resonance Energy Transfer (FRET) was used to characterise PAR₂ mediated ERK within different subcellular compartments upon stimulation with different agonists. The assays were repeated after employing pharmacological and genetic approaches shown to abolish receptor internalisation to assess the role of internalisation in generating compartmentalised signals.

### Methods

After achieving ˜50% confluency within 10 cm dishes HEK293 cells were transfected with 2-5 μg of PAR₂ with N-terminal HA epitope tag (PAR₂-HA) and 2-5 μg of desired FRET biosensor, using PEI as a transfection agent. 24 hours after transfection, cells were seeded into poly-D-lysine coated black 96 well isoplates. FRET was assessed 48 hours after cell plating, following overnight serum restriction. Before commencing FRET experiments, cell culture media was replaced with either 1×HBSS (+0.28 g HEPES/100 ml, pH 7.40); 30 μM Dyngo4a; 30 μM inactive Dyngo control; 30 μM PitStop2 or 30 μM inactive PitStop2 control. When used endocytic inhibitors were diluted in 1×HBSS+1% dimethyl sulfoxide (DMSO). Following the replacement of culture media cells were allowed to equilibrate at 37° C. for 30 minutes. FRET was then determined using a PHERAstar FS microplate reader (BMG LabTech, Offenburg, Germany), cells were sequentially excited at 430 nm with emissions being read at 550 nm and 490 nm. A 4 minute baseline was obtained before the addition of vehicle (1×HBSS+/−1% DMSO), protease or 1 μM PDBu (serving as a positive control). Measurements were then continued every minute for a further 30 minutes. FRET ratios were then calculated and corrected to baseline and vehicle treatments in Microsoft Excel 2013.

### Results

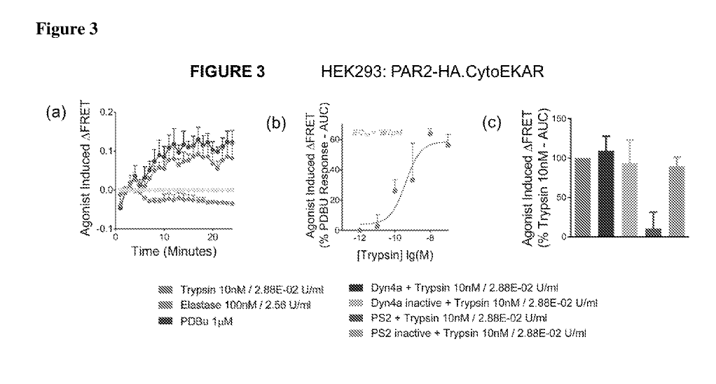

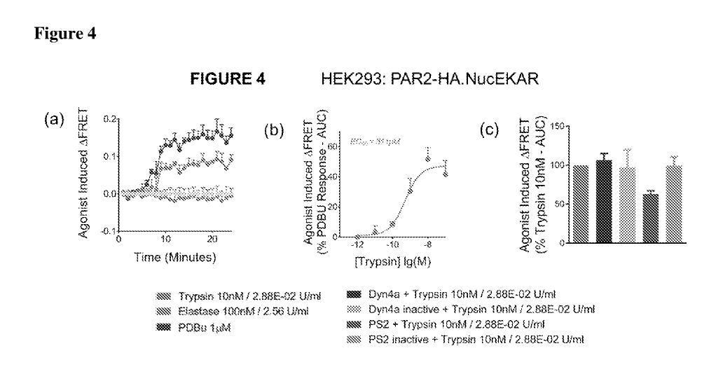

Trypsin, and not elastase, was able to produce ERK signals within the cytoplasm and nucleus (FIGS. 3a and 4a). Trypsin evoked ERK signals in these compartments were shown to be concentration dependent (FIGS. 3b and 4b). Cytosolic ERK phosphorylation is diminished when receptor internalisation is blocked through inhibition of clathrin with PS2 but not when dynamin is inhibited (FIG. 3c). Blocking internalisation through targeting of clathrin or dynamin does not seem to affect the extent of ERK phosphorylation in the nucleus (FIG. 4c).

### Example 3: BRET Resensitisation Assays

BRET resensitisation assays were used to quantify export of PAR₂ from the trans-golgi network (TGN38-venus) upon stimulation of receptor expressed at the cell surface with a variety of proteases. The assays were repeated with various inhibitors of signaling nodes to verify signaling mechanisms behind receptor resensitisation.

### Methods

After achieving ˜50% confluency within 10 cm dishes HEK293 cells were transiently transfected with PAR₂-RLuc8 (1 μg) and TGN38-Venus (4 μg) using PEI as a transfection agent. 24 hours post transfection, cells were seeded into poly-D-lysine coated 96-well isoplates. BRET experiments were conducted the following day. Cells were allowed to equilibrate in either 1×HBSS (+0-28 g HEPES/100 ml, pH 7-40), 10M Gallein (Gβγ inhibitor), 100 nM CRT0066101 (PKD inhibitor) or G06983 (PKC inhibitor) at 37° C. for 30 minutes before the addition of colenterazine H [5 μM]. Following a 5 minute incubation BRET was assessed using a LUMIstar Omega microplate reader (BMG LabTech, Offenburg, Germany), with luminescence being read at 535 nm and 475 nm. A 4 minute baseline was obtained before the addition of vehicle (1×HBSS) or protease, measurements were then continued for a further 30 minutes. BRET ratios were calculated using LUMIstar MARS analysis software and then corrected to baseline and vehicle treatments in Microsoft Excel 2013.

### Results

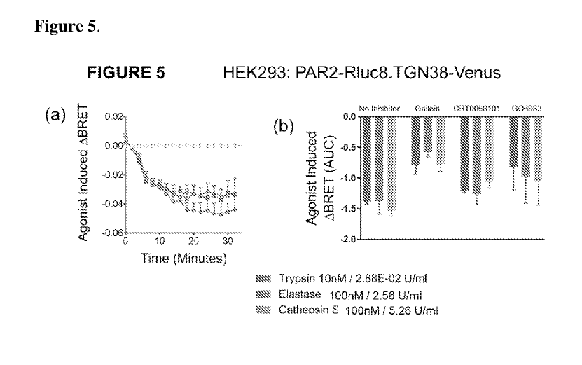

Luciferase tagged PAR₂ was shown to move away from the trans-golgi network upon stimulation of receptor expressed at the cell surface with all three agonists (FIG. 5a). These findings support PAR₂ resensitisation being achieved through receptor replenishment from a pre-existing pool of receptor stored within the trans-golgi network. The observed decreases in BRET were also found to be concentration dependent (data not shown).

Inhibition of Gβy with Gallein reduced PAR₂ trafficking away from the trans-golgi network upon stimulation with all three proteases, implicating this G-protein in the resensitisation process. Less prominent effects on normal trafficking were seen after inhibiting PKD and PKC with CRT0066101 and G06983 respectively.

### Example 4: Endocytic Inhibition of Neuronal Excitability

To assess the effects of proteases on excitability of nociceptors, DRG neurons were preincubated with trypsin, cathepsin S, elastase or vehicle (control), washed, and rheobase and action potential discharge at a current of twice rheobase were measured at 0 min or 30 min after washing.

### Methods

Isolation of DRG Neurons.

DRG (T9-T13) from C57BL/6 mice were digested by incubation in collagenase (1 mg/ml, Worthington Biochemical, Lakewood, N.J.) and dispase (4 mg/ml, Roche Life Science, Indianapolis, Ind.) for 10 min at 37° C., and were dispersed by trituration with a fire-polished Pasteur pipette. Cells were plated onto coverslips coated with laminin (0.017 mg/ml) and poly-D-lysine (2 mg/ml) in 24-well plates. Neurons were cultured in F12 medium (Cat N668, Lot RNBD5333, Sigma-Aldrich, St Louis, Mo. USA) containing 10% fetal calf serum, with penicillin and streptomycin and maintained at 37° C. in a humidified atmosphere of 95% air and 5% CO₂ until retrieval (16 h) for electrophysiological studies.

### Electrophysiology.

Neuronal excitability was assessed by perforated patch-clamp recordings with amphotericin B (240 μg/ml) from small diameter neurons (<30 pF capacitance) in current clamp mode at room temperature. Only neurons with resting membrane potentials more negative than −40 mV were analyzed. Changes in excitability were quantified by measuring rheobase (minimum current to fire action potential) and numbers of action potentials discharged at twice rheobase. Recordings were made using Axopatch 200B amplifiers, digitized by Digidata 1322A and stored and processed using pClamp 10.1 software (Molecular Devices, Sunnyvale, Calif.). The recording chamber was continuously perfused with external solution at 2 ml/min. External solution was (mM): 140 NaCl, 5 KCl, 10 HEPES, 10 D-glucose, 1 MgCl₂, 2 CaCl₂; pH to 7.4 with 3 M NaOH. Pipette solution was (mM): 110 K-gluconate, 30 KCl, 10 HEPES, 1 MgCl₂, 2 CaCl₂; pH 7.25 with 1 M KOH.

### Protease and Inhibitor Treatments.

DRG neurons were pre-incubated with vehicle (control) or proteases using conditions that we have reported to cause robust hyperexcitability: trypsin (50 nM, 10 min), elastase (10 U/ml, 390 nM, 30 min), Cat-S (500 nM, 60 min). Neurons were then washed and maintained in protease-free F12 medium. Neuronal excitability was assessed 0 or 30 min after washing. In some experiments, neurons were incubated with vehicle (0.3% DMSO), Dyngo4a (30 μM) or PitStop2 (15 μM) for 30 min before and during incubation with proteases.

### Statistical Analyses.

Results are expressed as mean±SE. Two way ANOVA and post hoc Tukey's tests were used to analyze the data.

### Results

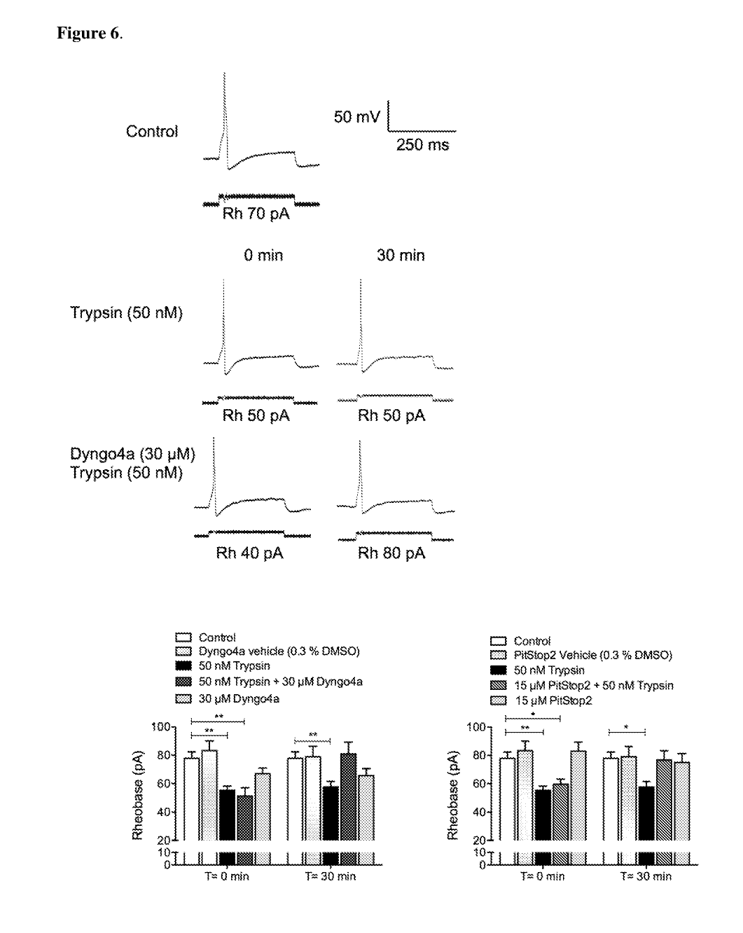

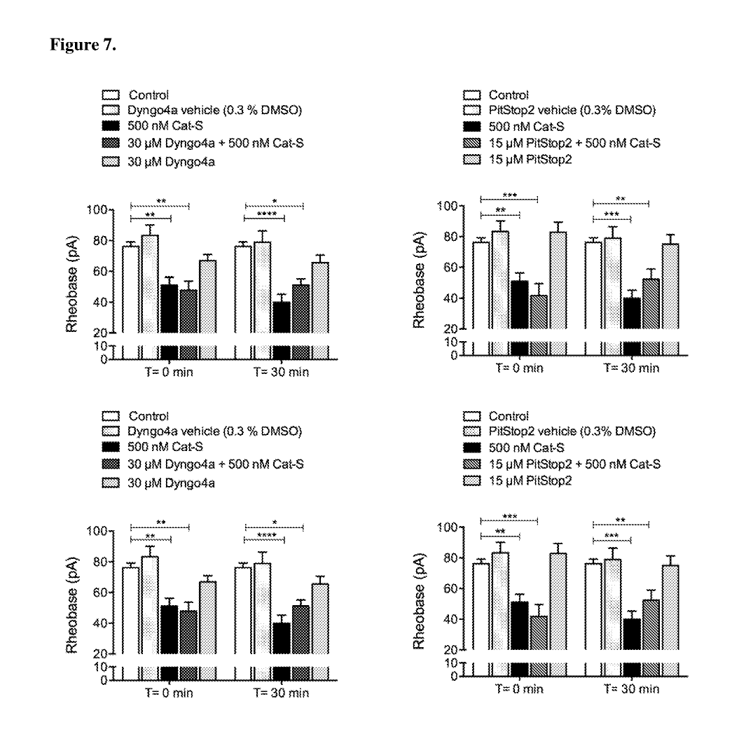

All proteases caused a decrease in rheobase at 0 min and 30 min, consistent with excitability. To assess the importance of clathrin and dynamin on excitability, DRG neurons were preincubated with the clathrin inhibitor PitStop2, the dynamin inhibitor Dyngo4a or vehicle (0.3% DMSO) 30 min before and during incubation with proteases. As can be seen in FIGS. 6 and 7, PitStop2 and Dymgo4a did not affect the effects of trypsin, cathepsin S or elastase on rheobase at 0 min. PitStop2 and Dyngo4a both prevented trypsin-induced decrease in rheobase at 30 min, but had no effect on cathepsin S- or elastase-induced decrease in rheobase at 30 min. Neither vehicle, PitStop2 nor Dyngo4a alone affected rheobase at 0 or 30 min.

### Example 5: Effects of Endocytic Inhibitors on Protease-Evoked Pain

### Methods

### Nociception Assays.

Immediately before experiments the mice were acclimatized to the experimental apparatus, room and investigator for 1-2 h on 2 successive days. Mechanical nociceptive responses were evaluated by paw withdrawal to stimulation of the plantar surface of the hind-paw with calibrated von Frey filaments. von Frey scores were measured in triplicate to establish a baseline for each animal on the day before experiments. Paw edema was estimated by measuring the thickness of the hindpaw using digital calipers before and after treatments. Mice were meditated with 5% isoflurane for intraplantar injections. Dyngo4a (50 μM), PitStop2 (50 μM) or vehicle (0.2% DMSO in 0.9% NaCl) (all 10 μl) were injected into the left hindpaw. After 30 min, trypsin (10 nM), Cat-S (2.5 μM) or elastase (30 U) (all 10 μl) were injected into the same left hindpaw. von Frey scores (left and right paws) and paw thickness (left paw) were measured hourly for up to 4 h after protease injection. Investigators were blinded to the test agents.

### Results

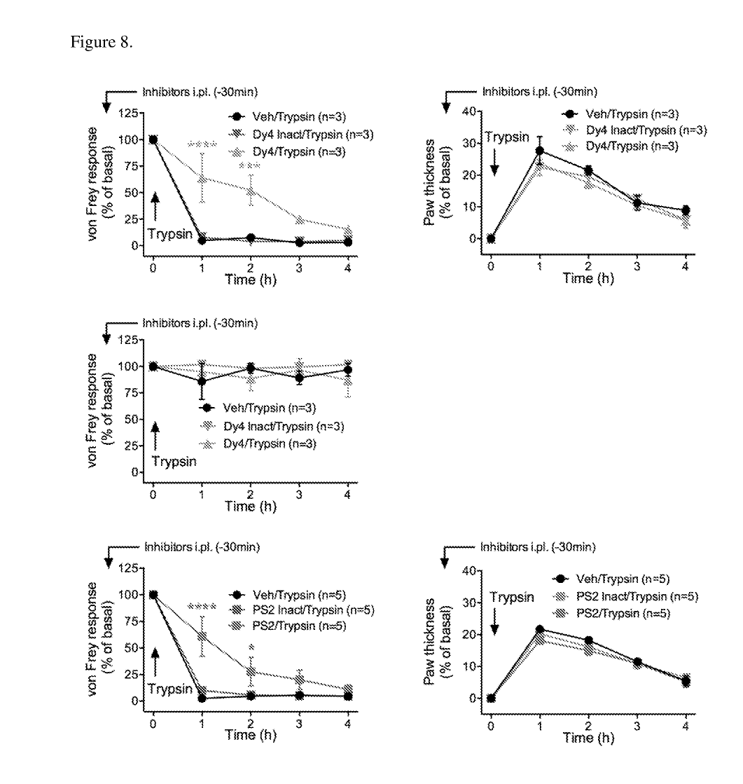

Intraplantar injection of trypsin, Cat-S and elastase caused mechanical hyperalgesia of the ipsilateral paw that was sustained for at least 4 h, and edema of the ipsilateral paw that was maximal after 4 h (FIGS. 8 & 9). It has previously been reported that these responses are PAR₂ and TRPV4 dependent. Dyngo and PitStop inhibited trypsin-evoked mechanical hyperalgesia. Dyngo did not affect Cat-S- or elastase-evoked mechanical hyperalgesia.

Dyngo did not affect trypsin-Cat-S- or elastase-evoked edema. The endocytic inhibitors did not affect withdrawal responses of the contralateral paw.

These results indicate that PAR₂ endocytosis is requited for central transmission of nociception induced by trypsin. PAR₂ endocytosis is not required for cathepsin S or elastase-evoked mechanical pain—these proteases do not cause PAR₂ endocytosis. PAR₂ endocytosis is not required for protease-evoked edema, which depends on Ca-mediated SP and CGRP release.

### Example 6: Inhibition of Endosomal PAR₂ Signaling

The inhibitory potency of various putative PAR₂ antagonists was determined in an assay measuring 2-furoyl-LIGRL-NH₂ (2F) stimulated IP₁ accumulation in KNRK cells stably expressing the human PAR₂ receptor or HT-29 cells that endogenously express PAR₂. Non-specific activity was determined in an assay measuring the ability of antagonists to inhibit ATP stimulated IP₁ accumulation in KNRK cells.

### Methods

KNRK-hPAR₂, KNRK or HT-29 cells were seeded at a density of 50×10³ cells/well in a clear poly-d-lysine coated 96 well tissue culture plate. Following 24 hrs incubation at 37° C. and 5% CO₂, media was removed and replaced with either 80l or 90l IP₁ stimulation buffer (10 mM HEPES, 1 mM CaCl₂, 0.5 mM MgCl₂, 4.2 mM KCl, 146 mM NaCl, 5.5 mM glucose, 50 mM LiCl). Following stimulation buffer addition, wells containing 80 μl buffer received 10 μl of 10× antagonists. All plates were further incubated at 37° C., 5% CO₂ for 30′. 10 μl of 2F or ATP was added to plates and further incubated for 40′. Following incubation, stimulation buffer was quickly removed by aspiration and replaced with 25 μl lysis buffer (IP-One HTRF® assay kit, Cisbio). Following incubation of the lysates at 37° C., 5% CO₂ for 10′, 10 μl lysate was transferred to a 384-well OptiPlate (PerkinElmer) and detected using the IP-One HTRF® assay kit (Cisbio).

Concentration response curve of PAR₂ antagonists ranging from 3 nM to 30M (added at 10× concentrations in a 10 μL volume) were incubated for 30 mins prior to the addition of 10 μM ATP or 100 nM or 300 nM 2F (EC₅₀, added at 10× concentrations in a 10 μL volume), and then further incubated for 40 min.

### Results

The PAR₂ antagonist illustrated below was found to inhibit 2F-stimulated IP₁ accumulation with an IC₅₀ of 5.39±0.13 μM.

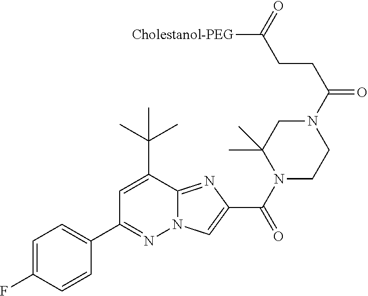

The antagonist did not have an effect on ATP-induced IP₁ accumulation in KNRK cells, suggesting that the antagonism of 2F in hPAR₂-KNRK cells is PAR₂ mediated. The antagonist neither had an effect on the baseline IP₁ accumulation in KNRK cells.

1. A method for the treatment of protease-evoked pain comprising administering to a subject in need thereof a compound that inhibits endosomal signaling of protease-activated receptor-2 (PAR₂).

2. The method according to claim 1 wherein the compound that inhibits endosomal signaling of PAR₂ is a compound that inhibits endocytosis of PAR₂.

3. The method according to claim 2, wherein the compound that inhibits endocvtosis of PAR₂ inhibits dynamin, clathrin or β-arrestin.

4. The method according to claim 1, wherein the compound that inhibits endosomal signaling of PAR₂ is a compound that targets and inhibits endosomal PAR₂ signaling.

5. The method according to claim 4, wherein the compound that targets and inhibits endosomal PAR₂ signaling is a tripartite compound of formula (I): 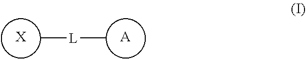
wherein
A is a lipid anchor that promotes insertion of the tripartite compound into a plasma membrane;
L is a linker moiety of 1 nm to 50 nm in length; and
X is an inhibitor of endosomal PAR₂ signaling;
wherein the lipid anchor partitions into lipid membranes that are insoluble in non-ionic detergent at 4° C.; or
a pharmaceutically acceptable salt thereof.

6. The method according to claim 5, wherein the tripartite compound of the formula (I) is represented by formula (Ia): 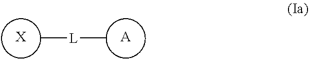
wherein
A is a lipid anchor that promotes insertion of the tripartite compound into a plasma membrane represented by formulae (II) or (III): 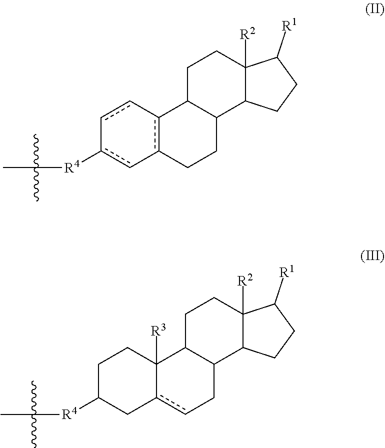
wherein
R¹ is an optionally substituted C₁₋₁₂ alkyl group;
R² and R³ are independently H or C₁₋₃alkyl;
R⁴ is CH₂, O, NH or S; and [symbol]
represents a single or double bond;
L is a linker group of 1 nm to 50 nm in length; and
X is an inhibitor of endosomal PAR₂ signaling; or
pharmaceutically acceptable salts thereof.

7. The method according to claim 5, wherein the tripartite compound of the formula (I) is represented by formula (Ib): 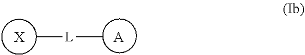
wherein
A is a lipid anchor that promotes insertion of the tripartite compound into a plasma membrane represented by formulae (II) or (III): 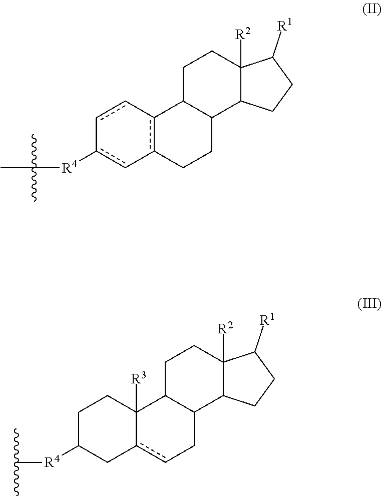
wherein
R¹ is an optionally substituted C₁₋₁₂ alkyl group;
R² and R³ are independently H or C₁₋₃alkyl;
R⁴ is CH₂, O, NH or S; [symbol]
represents a single or double bond;
L is represented by the formula (IV): 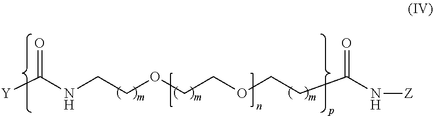
wherein
Z is the attachment group between the linker and the lipid anchor and is —C₁-C₁₀alkyl-, —C₂-C₁₀alkenyl-, —C₂-C₁₀alkynyl-, —C₁-C₁₀alkylC(O)—, —C₂-C₁₀alkenylC(O)— or —C₂-C₁₀alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
Y is the attachment group between the linker and the modulator of an endosomal GPCR and is —O—, —NH—, —S—, —C(O)—, —C(O)NH—, —C(O)O— or —C(O)S—; or
Y, together with the adjacent amido group is an amino acid selected from aspartic acid, glutamic acid, asparagine, glutamine, histidine, cysteine, lysine, arginine, serine or threonine; wherein the amino acid is attached to the modulator of an endosomal GPCR via its side-chain functional group;
m is 1 or 2;
n is from 1 to 20;
p is from 1 to 8; and
X is an inhibitor of endosomal PAR₂ signaling; or
pharmaceutically acceptable salts thereof.

8. The method according to claim 5, wherein the tripartite compound of the formula (I) has the structure: 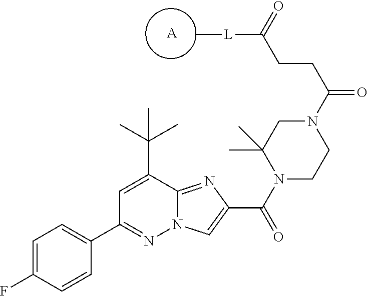
or a pharmaceutically acceptable salt thereof.

9. The method according to claim 1, comprising administering to the subject in need thereof a combination comprising one or more compounds that inhibit endocvtosis of PAR₂ and one or more compounds that target and inhibit endosomal PAR₂ signaling.

10.-17. (canceled)

18. The method according to claim 6, wherein the tripartite compound of the formula (I) has the structure: 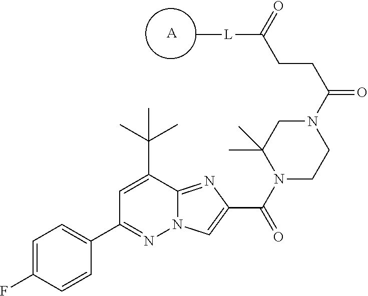
or a pharmaceutically acceptable salt thereof.

19. The method according to claim 7, wherein the tripartite compound of the formula (I) has the structure: 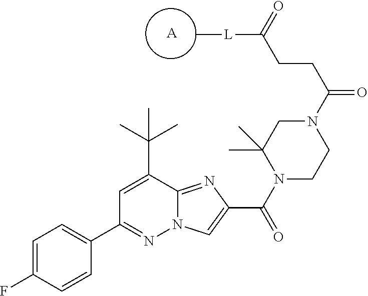
or a pharmaceutically acceptable salt thereof.

20. The method according to claim 1, wherein the compound that inhibits endosomal signaling of PAR₂ is a tripartite compound of formula (I): 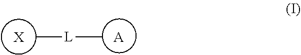
wherein
A is a lipid anchor that promotes insertion of the tripartite compound into a plasma membrane;
L is a linker moiety of 1 nm to 50 nm in length; and
X is an inhibitor of endosomal PAR₂ signaling;
wherein the lipid anchor partitions into lipid membranes that are insoluble in non-ionic detergent at 4° C.; or
a pharmaceutically acceptable salt thereof.

21. The method according to claim 20, wherein the tripartite compound of the formula (I) is represented by formula (Ia): 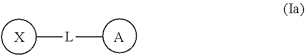
wherein
A is a lipid anchor that promotes insertion of the tripartite compound into a plasma membrane represented by formulae (II) or (III): 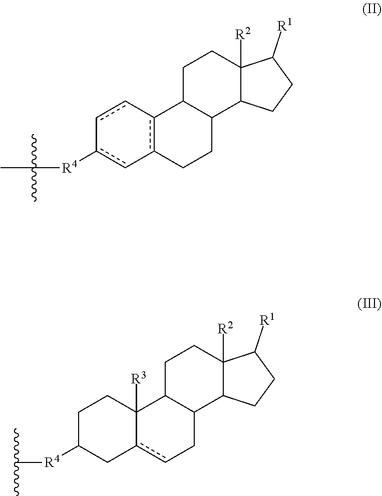
wherein
R¹ is an optionally substituted C₁₋₁₂ alkyl group;
R² and R³ are independently H or C₁₋₃alkyl;
R⁴ is CH₂, O, NH or S; and [symbol]
represents a single or double bond;
L is a linker group of 1 nm to 50 nm in length; and
X is an inhibitor of endosomal PAR₂ signaling; or
pharmaceutically acceptable salts thereof.

22. The method according to claim 20, wherein the tripartite compound of the formula (I) is represented by formula (Ib): 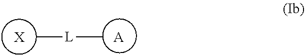
wherein
A is a lipid anchor that promotes insertion of the tripartite compound into a plasma membrane represented by formulae (II) or (III): 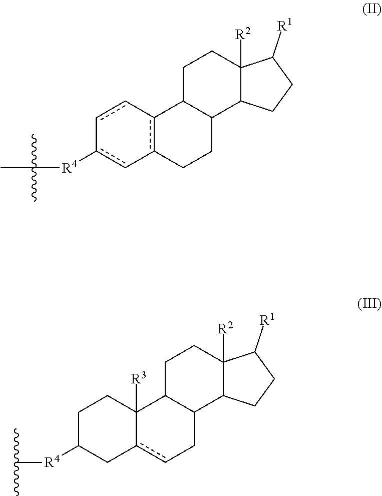
wherein
R¹ is an optionally substituted C₁₋₁₂ alkyl group;
R² and R³ are independently H or C₁₋₃alkyl;
R⁴ is CH₂, O, NH or S; [symbol]
represents a single or double bond;
L is represented by the formula (IV): 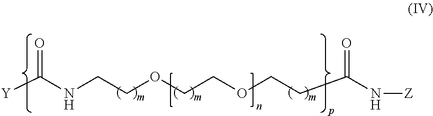
wherein
Z is the attachment group between the linker and the lipid anchor and is —C₁-C₁₀alkyl-, —C₂-C₁₀alkenyl-, —C₂-C₁₀alkynyl-, —C₁-C₁₀alkylC(O)—, —C₂-C₁₀alkenylC(O)— or —C₂-C₁₀alkynylC(O)—; or
Z, together with the adjacent amine, is an optionally C-terminal amidated amino acid selected from aspartic acid, glutamic acid, asparagine, glutamine, histidine, cysteine, lysine, arginine, serine or threonine; wherein the amino acid is attached to the lipid anchor via its side-chain functional group;
Y is the attachment group between the linker and the modulator of an endosomal GPCR and is —O—, —NH—, —S—, —C(O)—, —C(O)NH—, —C(O)O— or —C(O)S—; or
Y, together with the adjacent amido group is an amino acid selected from aspartic acid, glutamic acid, asparagine, glutamine, histidine, cysteine, lysine, arginine, serine or threonine; wherein the amino acid is attached to the modulator of an endosomal GPCR via its side-chain functional group;
m is 1 or 2;
n is from 1 to 20;
p is from 1 to 8; and
X is an inhibitor of endosomal PAR₂ signaling; or
pharmaceutically acceptable salts thereof.

23. The method according to claim 20, wherein the tripartite compound of the formula (I) has the structure: 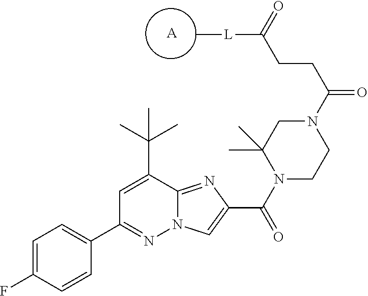
or a pharmaceutically acceptable salt thereof.

24. The method according to claim 21, wherein the tripartite compound of the formula (I) has the structure: 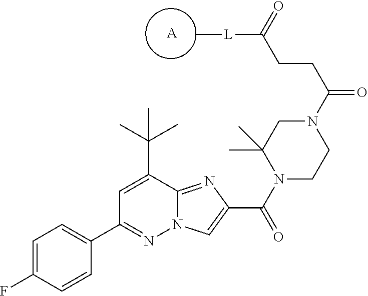
or a pharmaceutically acceptable salt thereof.

25. The method according to claim 22, wherein the tripartite compound of the formula (I) has the structure: 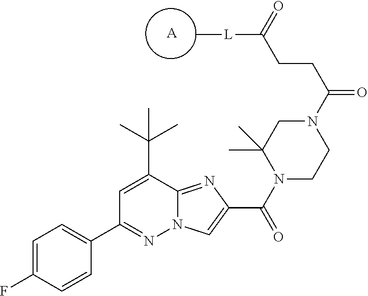
or a pharmaceutically acceptable salt thereof.
